(19) United States
(12) Patent Application Publication
(10) Pub. No.: US 2008/0217426 A1
(43) Pub. Date: Sep. 11, 2008

(54) Volatile Liquid Dissemination Apparatus

(75) Inventors: Colin Brown, Bracknell (GB); Guy Edward Naish, Bicester Oxfordshire (GB); Kishen Gohil, Surrey (GB)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 12/088,561

(22) PCT Filed: Sep. 26, 2006
(86) PCT No.: PCT/CH2006/000519

§ 371 Date: Mar. 28, 2008

(30) Foreign Application Priority Data
Sep. 29, 2005 (GB) 0519753.8

(51) Int. Cl. A61L 9/04

(52) U.S. Cl. 239/45

(57) ABSTRACT

An apparatus adapted to disseminate a volatile liquid, such as a fragrance, into an atmosphere in which its presence in vapour form is desired, the apparatus comprising a reservoir (1) of volatile liquid (2) and a porous wick that extends from the liquid in the reservoir to the atmosphere where it provides an evaporating surface, the wick being formed in at least two parts, at least one of which parts is in contact with the liquid and which comprises substantially no evaporating surface (3), and at least one other of which parts forms an evaporating surface (8,9), the parts being integral in the apparatus and that part comprising the evaporating surface being adapted to be movable either into liquid transfer contact or into non-liquid transfer separation with that part in contact with the liquid.

The apparatus is simple, rugged, effective, cheap to make and easy to use. It is especially useful as an air freshener in motor vehicles.

This invention relates to apparatus adapted to disseminate volatile liquids into an atmosphere.

Apparatus adapted to disseminate volatile liquids, such as fragrances, insecticides and fungicides, into an atmosphere are well known. One of the most common types involves the use of a porous wick that extends from a reservoir of volatile liquid, in which one end of the wick is immersed, into the atmosphere, where evaporation takes place from the other end. Such apparatus are simple and cheap, both to manufacture and operate, and they are reliable in use. However, in some applications, the basic simplicity can be a disadvantage. One such example is the use of such apparatus as air fresheners in motor vehicles. The main problem here is heat; a car left in the sun on a hot day can have interior temperatures exceeding 40° C. In such conditions, most or even all of the fragrance can be emitted, essentially rendering the apparatus ineffective and potentially generating an excess of fragrance & solvent in the confined space of the car interior.

There have been attempts to overcome this defect in automotive air fresheners. Some of these have involved using closable doors or louvres, operated by the rise in temperature, for example, by bimetallic strips. However, the friction involved in such moving parts has meant that this method is at best unreliable in operation. Also, unless these methods are entirely air-tight, fragrance will still evaporate from the exposed wick within the unit and the vapour escape through any slight gaps in the louvres.

It has now been found that it is possible to make a simple, reliable apparatus that overcomes the defects of the art referred to hereinabove. The invention therefore provides an apparatus adapted to disseminate a volatile liquid into an atmosphere in which its presence in vapour form is desired, the apparatus comprising a reservoir of volatile liquid and a porous wick that extends from the liquid in the reservoir to the atmosphere where it provides an evaporating surface, the wick being formed in at least two parts, at least one of which parts is in contact with the liquid and which comprises substantially no evaporating surface, and at least one other of which parts forms an evaporating surface, the parts being integral in the apparatus and that part comprising the evaporating surface being adapted to be movable either into liquid transfer contact or into non-liquid transfer separation with that part in contact with the liquid.

The invention further provides a method of controlling dissemination of a volatile liquid from an apparatus having a porous wick through which the liquid is transferred from a reservoir to an evaporating surface forming part of the wick, comprising providing a wick that is in at least two parts, at least one part being in contact with the liquid and having substantially no evaporating surface, and at least one part providing an evaporating surface, the parts being separable, and causing the part in contact with the liquid and the part with the evaporating surface to come into liquid transfer contact or non-liquid transfer separation, as desired.

The basis of this invention is a multi-part wick. It will now be generally described with reference to the most common embodiment, a two-part wick, one in contact with the liquid and one with an evaporating surface. In addition, explicit reference will be made to embodiments in which there are more than two parts.

Apart from the nature of the wick and any construction required for the performance of its function, the apparatus according to the invention is basically the same as other known wick-based dissemination apparatus, and insofar as the apparatus of the present invention has common details, the same materials and construction techniques may be used.

That part of the wick that is in contact with the liquid in the reservoir (hereinafter “the lower wick”) provides substantially no evaporation surface. The evaporation surface provided by a wick is generally provided by that part that extends outside the reservoir into the atmosphere. Thus, that part of the lower wick extending into the atmosphere is either kept as short as possible, or it is prevented in some way from providing an evaporating surface. The most usual way is to provide a lower wick that does not extend into the atmosphere at all, that is, that end remote from the liquid either is flush with the exterior of the reservoir (usually with the surface of a cap into which the wick fits), or it is recessed into the reservoir. This means that only a cross-section of the lower wick is exposed to the atmosphere and this is effectively too small to provide an evaporation surface (evaporation is negligible).

Should the lower wick extend from the reservoir into the atmosphere, it can be prevented from providing an evaporation surface by providing any potential evaporation surface with an impermeable coating. This can be, for example, a solidified liquid coating composition applied to the surface, or a sleeve of solid, impermeable material fitted to the otherwise exposed surface of the lower wick.

That part of the wick providing the evaporation surface (hereinafter “the upper wick”), is integral with the apparatus, that is, in its operational form, it forms part of a single apparatus. The apparatus may come in parts and require assembly prior to use, but in general the apparatus comes already assembled and ready for use, and the component parts are not readily detachable or removable therefrom. The nature of this integral nature will vary depending on the precise nature of the apparatus, but in general the upper wick is held in place by the means that allows it to be moved. For example, the upper wick may be slidably mounted, for example, in a sleeve or other retaining means, allowing it to be moved, yet remain part of the apparatus. Other possibilities and variants will be clear to the skilled person and some of these will be further discussed hereinunder.

The upper wick is located in the apparatus such that it can be moved into or out of liquid transfer contact with the lower wick. By “liquid transfer contact” is meant simply that the upper and lower wicks can be brought into contact such that liquid can flow from the lower wick to the upper wick. This can be simply the abutting of two flat ends of the upper and lower wicks. These ends may also be shaped in any suitable way, or one end may penetrate into the other, for example, by one having an elongate projection and the other end having a matching orifice into which it fits.

Although, for reasons of cost and convenience, the upper wick is usually of the same material and same diameter as the lower wick, this need not necessarily be the case, and in some embodiments of the invention, the upper wick is of a different material and/or shape. This may be done, for example, to provide the upper wick with a greater evaporating surface or an enhanced evaporating ability.

The movement of the upper wick in and out of liquid transfer contact with the lower wick may be achieved manually. For example, as previously described, the upper wick may be slidably mounted, for example, in a sleeve within which it is slidable towards and away from the lower wick in a linear fashion. To achieve liquid transfer contact, the upper wick may be slid into contact and out of contact again. In one embodiment, the upper wick may comprise a number of separate wicks, which may be moved independently of each other. For example, the upper wick may comprise two or more wick elements that fit side-by-side within the same sleeve, are retained therein and can slide past one another. This allows for control of rate of emission of liquid, depending one whether one or more wick elements are in liquid transfer contact with the lower wick.

In another embodiment of manual movement, the upper wick may be rotated into and out of liquid transfer contact with the lower wick. There are many ways of achieving this that are within the skill of the art, but in a typical example at least one upper wick is mounted in a rotatable frame, which is arranged such that rotation will bring the upper wick into contact with the lower wick. Further rotation will remove the contact. Two or more upper wicks may be used, and it is possible to make these wicks different in form or material, such that the individual wicks have different emission characteristics that can allow for different atmospheric conditions, for example, slower emission characteristics for warmer days and faster emission characteristics for cooler days.

In a preferred embodiment, the achievement of liquid transfer contact and non-liquid transfer separation is achieved automatically. A preferred means of achieving this is by means of the attachment of the upper wick to a heat-sensitive bimetallic element that in one temperature position holds the upper and lower wicks in liquid transfer contact, and in another temperature position holds them in non-liquid transfer separation. Such a bimetallic element, typically a spring, will generally be configured such that liquid transfer contact is made at low temperatures and non-liquid transfer separation is made at elevated temperature. Such a system is particularly useful in the case of an automotive air fragrance; in such a case, the emission of fragrance will be stopped when the temperature inside the vehicle rises, and it will be resumed when it cools down again The bimetallic element may have the additional function of securing the upper wick in place on the apparatus.

In the bimetallic element case, the upper wick may comprise two or more elements, each of which is secured by and acted upon by its own bimetallic element. Such bimetallic elements can be selected such that they cause separation of the upper wick element to which they are attached from the lower wick at different temperatures. Thus, the quantity of emission can be controlled more precisely and each of the wick elements breaks liquid transfer contact sequentially as the temperature rises.

The apparatus of the invention is simple to make, rugged and reliable in operation and offers considerable versatility over the previously-known and -used wick-based systems, especially those used in automotive air fresheners.

The invention is now further described with reference to the drawings, which depict preferred embodiments of an air freshener, and which are not intended to be in any way limiting. The skilled person will readily comprehend that there are many variants of this invention lying within the skill of the art, but all of which lie within the scope of this invention.

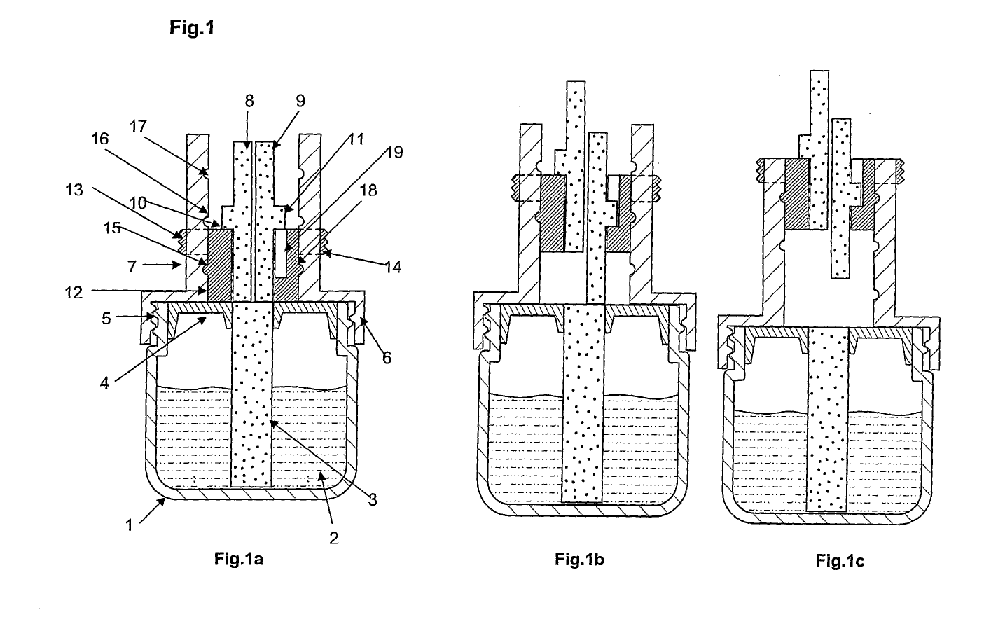

FIG. 1 is a schematic vertical cross-section through a manually-operated embodiment according to the invention, showing three aspects of its operation.

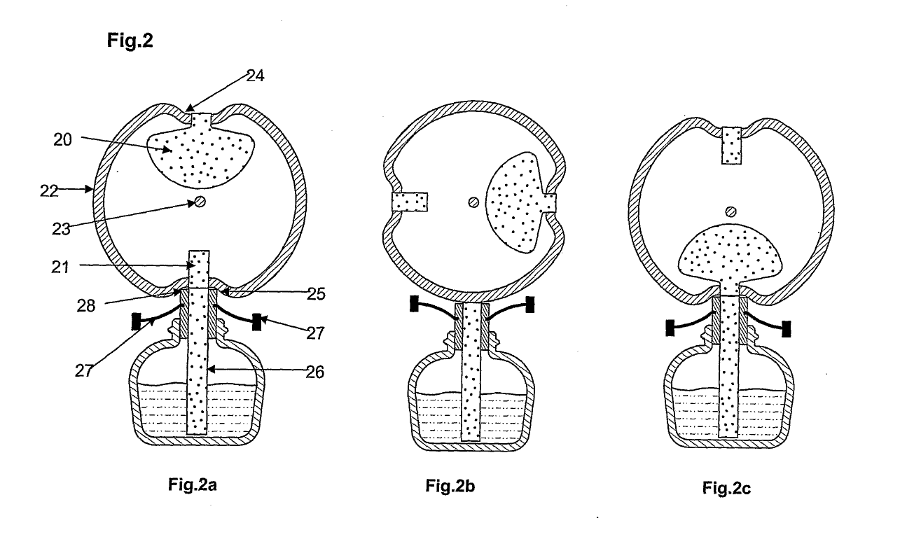

FIG. 2 is a schematic vertical cross-section through a rotary manually-operated embodiment according to the invention, showing three aspects of its operation.

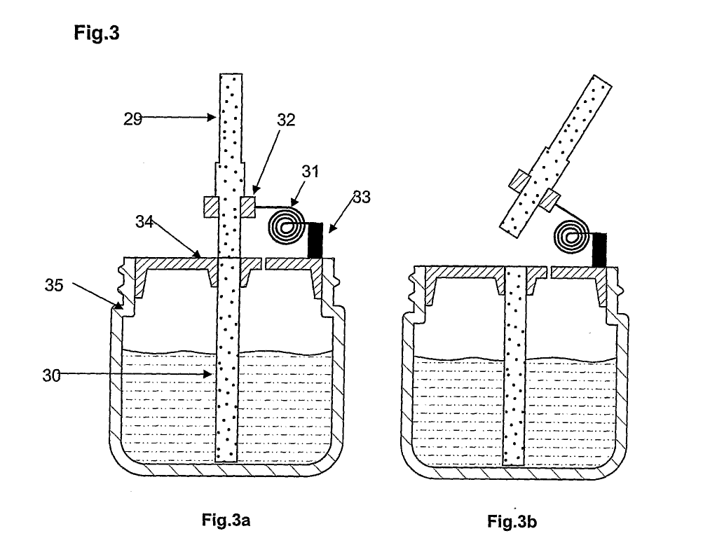

FIG. 3 is a schematic vertical cross section through an automatic embodiment, showing two aspects of its operation.

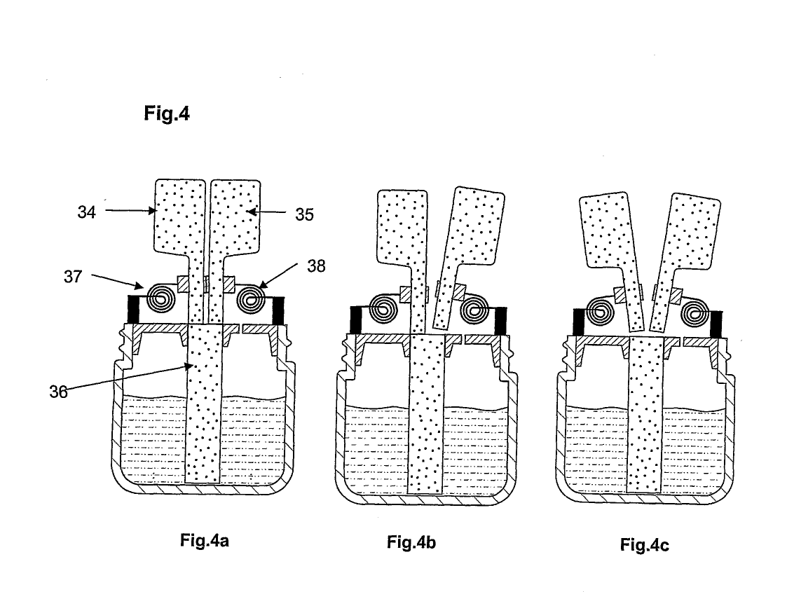

FIG. 4 is a schematic vertical cross-section through a variant of the embodiment of FIG. 2, showing three aspects of its operation.

In FIGS. 1a-1c, a reservoir 1 contains a volatile liquid 2. In contact with the liquid is a lower cylindrical wick 3, held in place by an inner cap 4 within which it is a tight fit. The upper end of the lower wick 3 ends flush with the outer surface of the inner cap 4. Attached to the reservoir by cooperating screw threads 5 is an outer cap 6. This outer cap has a cylindrical neck portion 7 extending upwards from the reservoir. Within this neck are placed two wick elements 8, 9, which, when placed together side-by side, have the general form of a cylinder of the same diameter as that of the lower wick 3.

These wick elements 8, 9 additionally comprise projections 10, 11, whose functions are to permit the moving of the wick elements. This movement is achieved by a sliding element 12, which has the basic form of a cylinder and which is slidable within the neck 7, movement being effected by tabs 13, 14 projecting from the neck through elongate slits extending along the neck away from the reservoir. Within the neck are three circumferential grooves, a lower groove 15, a middle groove 16 and an upper groove 17. Into these grooves can fit a correspondingly-shaped circumferential ridge 18 on the outer surface of the sliding element 12. The grooves thus define three positions in which the sliding element 12, and therefore the wick elements 8, 9, can be held. The neck 7 and/or the sliding element 12 are made of a material that is sufficiently resilient to allow the sliding element to be moved, yet hold it fast when the ridge is in one of the grooves 15-17.

The sliding element 12 additionally comprises a groove 19, whose transverse cross-sectional shape matches, and is aligned with, that of the wick element projection 11, and dimensioned such that the sliding element 12 does not come into wick element-raising contact with the projection 11 until the ridge 18 is in the middle groove 16. In contrast, the other wick element projection 10 is already in wick element-raising contact with the sliding element when the ridge 18 is in the lower groove 15.

In FIG. 1a, both wick elements 8, 9 are in liquid transfer contact with the lower wick 3. In FIG. 1b, there is shown the effect of moving the sliding element 12 away from the reservoir, such that the ridge 18 moves from the lower groove 15 to the middle groove 16. The sliding element 12 pushes immediately against the projection 10, raising the wick element 8 away from and out of liquid transfer contact with the lower wick 3. On the other hand, the other wick projection 11 slides within the groove 19 and remains in contact with the lower wick until the ridge 18 comes into register with the middle groove 16. This defines an intermediate state of operation, in which only one wick element emits fragrance, and the amount in the atmosphere is thus reduced.

In FIG. 1c, the sliding element 12 is pushed further away from the reservoir, until the ridge 18 comes into register with the upper groove 17. When this happens, the sliding element pushes on both projections 10 and 11, and both wick elements are pushed out of contact with the lower wick 3. In this position, there is no emission of fragrance from the apparatus.

In FIGS. 2a-2b, two upper wicks 20, 21 of different shapes are held in a frame 22, which rotates in the plane of the page about a pivot 23. The frame is basically circular, with the wicks being held in re-entrant portions 24, 25 of the frame, such that the ends of the wicks that will come into liquid transfer contact with a lower wick 26 lie closer to the centre of the circle made by the frame. The holding structure of the rotating frame and the reservoir (not shown) includes leaf spring elements 27 attached to the neck 28 of the reservoir. The rest position of these spring elements occurs when the upper and lower wicks are in contact. This means that, when the upper and lower wicks are not in contact, the spring elements are under compression, and when the frame is rotated such that a re-entrant portion 24 or 25 is opposite the lower wick, the spring elements urge the two wicks into contact. The profiles of the re-entrant portions are shaped such that they act as cam profiles when the frame is turned, forcing the upper and lower wicks apart against the force of the spring elements. In the drawings of FIGS. 2a-2c, the two upper wicks 20, 21 have different shapes, wick 20 having a much bigger surface area than wick 21. This means that the emission of fragrance from wick 20 will be substantially greater than that from wick 21. Thus, the amount of fragrance emission can be regulated, by selecting which upper wick is in contact with the lower wick.

In FIGS. 3a-3b, the element that both connects an upper wick 29 to an apparatus and maintains it in liquid transfer contact with, or moves it into non-liquid transfer separation from, a lower wick 30 is a bimetallic spring 31. This is attached by means of a sleeve 32 to the upper wick 29 and by a mounting element 33 fixed to a cap 34 that closes an open end of a reservoir 35. The spring is configured such that, at low temperatures, the upper wick 29 is in liquid transfer contact with the lower wick 30, and at more elevated temperatures, the spring moves the upper wick away from contact with the lower wick.

FIGS. 4a-4c depicts a version of the apparatus of FIGS. 3a-3c, in which the upper wick 29 of FIG. 3 is replaced by a double upper wick composed of two wick elements 34, 35 in liquid transfer contact with a lower wick 36. Each wick element has its own bimetallic spring 37, 38, which springs work independently of each other. These springs are selected and arranged such that wick element 35 is removed from liquid transfer contact with lower wick 36 at an intermediate temperature and wick element 36 is removed from liquid transfer contact with lower wick 36 at a higher temperature. This provides, in essence, an automatic version of the embodiment of FIGS. 2a-2c

1. An apparatus adapted to disseminate a volatile liquid into an atmosphere in which its presence in vapour form is desired, the apparatus comprising a reservoir of volatile liquid and a porous wick that extends from the liquid in the reservoir to the atmosphere where it provides an evaporating surface, the wick being formed in at least two parts, a lower wick in contact with the liquid and which comprises substantially no evaporating surface, and an upper wick which forms an evaporating surface the parts being integral in the apparatus and the upper wick being adapted to be movable either into liquid transfer contact or into non-liquid transfer separation with the lower wick.

2. An apparatus according to claim 1, in which the upper wick is slidable in and out of liquid transfer contact with the lower wick.

3. An apparatus according to claim 1, in which the upper wick is rotatable in and out of liquid transfer contact with the lower wick.

4. An apparatus according to claim 3, in which the rotatable wick is mounted in a rotatable frame.

5. An apparatus according to claim 1, in which the making and breaking of liquid transfer contact is achieved by automatic means.

6. An apparatus according to claim 5, in which the automatic means comprises a heat-sensitive bimetallic element.

7. An apparatus according to claim 1, in which the upper wick comprises a number of separate wicks.

8. An apparatus according to claim 6, in which a plurality of upper wicks is caused to make and break liquid transfer contact by means of bimetallic springs attached to the individual upper wicks.

9. A method of controlling dissemination of a volatile liquid from an apparatus having a porous wick through which the liquid is transferred from a reservoir to an evaporating surface forming part of the wick, comprising providing a wick that is in at least two parts, at least one part being in contact with the liquid and having substantially no evaporating surface, and at least one part providing an evaporating surface, the parts being separable, and causing the part in contact with the liquid and the part with the evaporating surface to come into liquid transfer contact or non-liquid transfer separation, as desired.
